(19) United States
(12) Patent Application Publication
(10) Pub. No.: US 2005/0042618 A1
(43) Pub. Date: Feb. 24, 2005

(54) Fluorescent resonance energy transfer probes

(75) Inventors: Dieter Heindl, Paehl (DE); Hans-Peter Josel, Weilheim (DE); Gregor Sagner, Penzberg (DE); Ingrid Bechler, Geretsried (DE)

Correspondence Address: ROCHE MOLECULAR SYSTEMS INC;PATENT LAW DEPARTMENT, 1145 ATLANTIC AVENUE, ALAMEDA, CA, 94501, US

(73) Assignee: ROCHE MOLECULAR SYSTEMS, INC., Alameda, CA (US)

(21) Appl. No.: 10/635,171

(22) Filed: Aug. 6, 2003

(30) Foreign Application Priority Data
Aug. 6, 2002 (EP) 02017629.3

(51) Int. Cl. C12Q 1/68; C07H 21/04

(52) U.S. Cl. 435/6; 536/24.3; 536/25.32

(57) ABSTRACT

Compositions, reaction mixtures, and kits, as well as methods for their use, comprising a pair of FRET hybridization probes hybridizing adjacently to a target nucleic acid sequence, each hybridization probe comprising (i) a nucleotide sequence entity which is substantially complementary to the sequence of the target nucleic acid, (ii) a fluorescent entity, being either a FRET donor entity or a FRET acceptor entity, and (iii) a spacer entity connecting the nucleotide sequence entity and the fluorescent entity. The intensity of fluorescence emission from the FRET donor entity and from the FRET acceptor entity is not substantially affected by quenching activity of nucleotide residues present in the sequence of the target nucleic acid or in the nucleotide sequence entities of the hybridization probes.

### BACKGROUND OF THE I INVENTION

1. Field of the Invention

The subject of this application relates to real time (kinetic) PCR. More specifically, the subject relates to an improved design of FRET hybridization probes.

2. Description of Related Art

In kinetic real-time PCR, the formation of PCR products is monitored in each cycle of the PCR. The amplification is usually measured in thermocyclers which have additional devices for measuring fluorescence signals during the amplification reaction. A typical example of this is the Roche Diagnostics LightCycler (Cat. No. 2 0110468). The amplification products are for example detected by means of fluorescent labelled hybridization probes which only emit fluorescence signals when they are bound to the target nucleic acid or in certain cases also by means of fluorescent dyes that bind to double-stranded DNA. A defined signal threshold is determined for all reactions to be analysed and the number of cycles Cp required to reach this threshold value is determined for the target nucleic acid as well as for the reference nucleic acids such as the standard or housekeeping gene. The absolute or relative copy numbers of the target molecule can be determined on the basis of the Cp values obtained for the target nucleic acid and the reference nucleic acid (Roche Diagnostics LightCycler operator manual(Cat. No. 2 0110468))

The FRET hybridization probe test format, which may be used in real time PCR, is characterized by two single-stranded hybridization probes which are used simultaneously and are complementary to adjacent sites of the same strand of the amplified target nucleic acid. Both probes are labeled with different fluorescent components. When excited with light of a suitable wavelength, a first component transfers the absorbed energy to the second component according to the principle of fluorescence resonance energy transfer such that a fluorescence emission of the second component can be measured when both hybridization probes bind to adjacent positions of the target molecule to be detected. Alternatively, fluorescence decrease of the FRET donor component may be monitored. Among all detection formats known in the art, this FRET-hybridization probe format has been proven to be highly sensitive, exact and reliable (WO 97/46707; WO 97/46712; WO 97/46714. The design of appropriate FRET hybridization probe sequences may sometimes be limited by the special characteristics of the target nucleic acid sequence to be detected.

Alternatively, it is also possible to use a fluorescent-labeled primer and only one labeled oligonucleotide probe (Bernard, P. S., et al., Anal Biochem 255 (1998) 101-7.).

Another application of FRET hybridization probes is melting curve analysis. In such an assay, the target nucleic acid is amplified first in a typical PCR reaction with suitable amplification primers. The hybridization probes may already be present during the amplification reaction or added subsequently. After completion of the PCR-reaction, the temperature of the sample is constitutively increased, and fluorescence is detected as long as the hybridization probe was bound to the target DNA. At melting temperature, the hybridization probes are released from their target, and the fluorescent signal is decreasing immediately down to the background level. This decrease is monitored with an appropriate fluorescence versus temperature-time plot such that a first derivative value can be determined, at which the maximum of fluorescence decrease is observed.

However, for kinetic real time PCR as well as for melting curve analysis, tremendous differences in absolute signal intensities have been observed for different pairs of FRET hybridization probes, although being labeled with the same couple of fluorescent dyes. Moreover, this phenomenon is independent from the couple of fluorescent dyes which is used.

The reason for the observed effect is unknown, although one may speculate that it could be due to quenching or dequenching effects of G residues which have been disclosed previously in various systems (WO 01/36668, Seidel, C. A. M., et al., J Phys Chem 100 (1996) 5541-53, 1996).

In most cases, G residues causing quenching effects are usually located in close spatial vicinity to the respective fluorescent compound (EP 1 046 717). Moreover, based on this effect, it is was possible in some cases to set up an assay, wherein fluorescent emission of an unhybridized labeled probe is quenched by internal residues and hybridization can monitored due to a dequenching effect occurring as soon as the probe is being hybridized to a complementary target sequence (WO 01/73118).

Due to the unpredictable overall structures of nucleic acids, however, G residues causing quenching effects can be located at different positions in the target DNA as well as in the hybridization probes themselves.

In any case, the observed effect is highly disadvantageous especially with respect to the design of multiplex assays, characterized in that within one reaction vessel, one or more target sequences are amplified and quantitatively analyzed with two or multiple pairs of FRET hybridization probes. Thus there is a need in the art for an improved design of FRET hybridization probes wherein the absolute signal is not affected by the base composition and sequence of the target nucleic acid.

### SUMMARY OF THE INVENTION

In one aspect, there are provided compositions, reaction mixtures and kits comprising a pair of FRET hybridization probes, wherein the emission signals of the fluorescent moieties are not interfered with by any quenching activity of any nucleotide residue in the vicinity of the fluorescent moieties.

More precisely, a pair of FRET hybridization probes hybridizes adjacently to a target nucleic acid sequence, each hybridization probe comprising

a nucleotide sequence entity which is substantially complementary to the sequence of the target nucleic acid;
a fluorescent entity, the entity being either a FRET donor entity or a FRET acceptor entity, and
a spacer entity connecting the nucleotide sequence entity and the fluorescent entity,
   wherein the spacer entities of the two members of the pair of FRET hybridization probes are capable of forming noncovalent interactions with each other. The noncovalent interactions may be, for example, nucleotide base pairing interactions, and preferably A/T base pairing interactions.

In another aspect, there are provided compositions, reaction mixtures and kits comprising a pair of FRET hybridization probes hybridizing adjacently to a target nucleic acid sequence, each hybridization probe comprising
a nucleotide sequence entity which is substantially complementary to the sequence of the target nucleic acid;
a fluorescent entity, the entity being either a FRET donor entity or a FRET acceptor entity; and
a spacer entity connecting the nucleotide sequence entity and the fluorescent entity.
   In another aspect, there are provided methods for use of the compositions, reaction mixtures and kits described herein.

As will be shown in the Examples, due to the presence of the spacer entities, the intensity of fluorescence emission from the FRET donor entity and the intensity of fluorescence emission from the FRET acceptor entity are not substantially affected by any quenching activity of nucleotide residues present in the sequence of the target nucleic acid or present in the nucleotide sequence entities of the hybridization probes themselves.

### DESCRIPTION OF THE FIGURES

FIG. 1: graphs of fluorescence versus cycle number plot of the real time PCR experiment of Example 2 using fluorescein/JA286-labeled FRET hybridization probes to detect a FactorV amplicon. +Stem: FRET hybridization probes carrying a stem−Stem: FRET hybridization probes without stem. FIG. 1A=10⁶ copies of target DNA; FIG. 1B=10⁴ copies of target DNA.

FIG. 2: graphs showing the 1^{st} derivatives of fluorescence versus temperature plot showing the melting curve analysis of the experiment disclosed in Example 3 using fluorescein/JA286 FRET hybridization probes. +Stem: FRET hybridization probes carrying a stem−Stem: FRET hybridization probes without stem. FIG. 2A=10⁶ copies of target DNA; FIG. 2B=10⁴ copies of target DNA.

FIG. 3: graphs showing fluorescence versus cycle number plot of the real time PCR experiment disclosed in Example 4 using fluorescein/LC-Red-640 FRET hybridization probes to detect a Factor V amplicon. +Stem: FRET hybridization probes carrying a stem−Stem: FRET hybridization probes without stem. FIG. 3A=10⁶ copies of target DNA; FIG. 3B=10⁴ copies of target DNA.

FIG. 4: graphs of 1^{st} derivatives of fluorescence versus temperature plot showing the melting curve analysis of the experiment disclosed in Example 5 using fluorescein/LC-Red-640 FRET hybridization probes. +Stem: FRET hybridization probes carrying a stem−Stem: FRET hybridization probes without stem. FIG. 4A=10⁶ copies of target DNA; FIG. 4B=10⁴ copies of target DNA.

FIG. 5: graph showing fluorescence versus cycle number plot of the real time PCR experiment disclosed in Example 6 using fluorescein/JA286 FRET hybridization probes to detect a G6PDH amplicon. +Stem: FRET hybridization probes carrying a stem−Stem: FRET hybridization probes without stem. FIG. 5A=10⁸ copies of target DNA; FIG. 5B=10³ copies of target DNA.

FIG. 6: graph showing fluorescence versus cycle number plot of the real time PCR experiment disclosed in Example 7 using fluorescein/JA286 FRET hybridization probes to detect a FactorV amplicon comprising different A/T stems. Without A/T=FRET hybridization probe carrying no stem; 1 A/T=FRET hybridization probe carrying a 1 base pair A/T stem; 3 A/T=FRET hybridization probe carrying a 3 base pair A/T stem; 5 A/T=FRET hybridization probe carrying a 5 base pair A/T stem.

FIG. 7: graph showing 1^{st} derivatives of fluorescence versus temperature plot showing the melting curve analysis of the experiment disclosed in Example 8 using fluorescein/JA286 FRET hybridization probes comprising different A/T stems. Without A/T=FRET hybridization probe carrying no stem; 1 A/T=FRET hybridization probe carrying a 1 base pair A/T stem; 3 A/T=FRET hybridization probe carrying a 3 base pair A/T stem; 5 A/T=FRET hybridization probe carrying a 5 base pair A/T stem.

### DETAILED DESCRIPTION OF THE INVENTION

The FRET hybridization probe test format is characterized by two single-stranded hybridization probes which are used simultaneously and are complementary to adjacent sites of the same strand of the amplified target nucleic acid. Both probes are labeled with different fluorescent components. When excited with light of a suitable wavelength, a first component transfers the absorbed energy to the second component according to the principle of fluorescence resonance energy transfer such that a fluorescence emission of the second component can be measured when both hybridization probes bind to adjacent positions of the target molecule to be detected.

The desired effect of preventing any quenching effect is achieved by a pair of FRET hybridization probes hybridizing adjacently to a target nucleic acid sequence, each hybridization probe comprising
a nucleotide sequence entity which is substantially complementary to the sequence of the target nucleic acid
a fluorescent entity, said entity being either the FRET donor entity or the FRET acceptor entity
a spacer entity connecting said nucleotide sequence entity and said fluorescent entity,
   wherein said spacer entities of the two members of said pair of FRET hybridization probes are capable of forming non covalent interactions with each other.

In addition to the desired effect of prevention of quenching effects, such an inventive pair of FRET hybridization probes has further advantages. First, the kinetics of formation of the ternary complex composed of the target nucleic acid and the two members of the pair of hybridization probes may be accelerated. Second, the melting temperature of the FRET hybridization probes in some instances may be altered at least slightly. In certain cases, such an adjustment may be required for an optimal design of a multiplex assay.

As used herein, the term “spacer entity” is a chemical linker structure with a molecular weight of at least 300.

In one aspect, provided herein is a composition comprising a pair of FRET hybridization probes capable of hybridizing to a target nucleic acid sequence, each of said pair of FRET hybridization probe comprising a nucleotide sequence entity, said nucleotide sequence entity being substantially complementary to a portion of the sequence of the target nucleic acid; a fluorescent entity, said fluorescent entity being either a FRET donor entity or a FRET acceptor entity; and a spacer entity, said spacer entity connecting the nucleotide sequence entity and the fluorescent entity; wherein the FRET hybridization probes hybridize adjacently to each other on the target nucleic acid; and wherein the spacer entities of the FRET hybridization probes are capable of forming non covalent interactions with each other.

In another aspect, there is provided a kit for use in performing a template dependent nucleic acid amplification reaction, comprising a pair of hybridization probes capable of hybridizing to a target nucleic acid sequence, each of said pair of FRET hybridization probe comprising a nucleotide sequence entity, said nucleotide sequence entity being substantially complementary to a portion of the sequence of the target nucleic acid; a fluorescent entity, said fluorescent entity being either a FRET donor entity or a FRET acceptor entity; and a spacer entity, said spacer entity connecting the nucleotide sequence entity and the fluorescent entity; wherein the FRET hybridization probes hybridize adjacently to each other on the target nucleic acid; and wherein the spacer entities of the FRET hybridization probes are capable of forming non covalent interactions with each other; at least one other component selected from the group consisting of nucleic acid amplification primers, a template dependent nucleic acid polymerase, deoxynucleoside triphosphates and a buffer suitable for use in a template dependent nucleic acid amplification reaction; and a container.

In another aspect, there is provided a method for performing a real-time template dependent nucleic acid amplification reaction, comprising contacting a sample containing a target nucleic acid sequence with a pair of hybridization probes capable of hybridizing to a target nucleic acid sequence, each of said pair of FRET hybridization probe comprising a nucleotide sequence entity, said nucleotide sequence entity being substantially complementary to a portion of the sequence of the target nucleic acid; a fluorescent entity, said fluorescent entity being either a FRET donor entity or a FRET acceptor entity; and a spacer entity, said spacer entity connecting the nucleotide sequence entity and the fluorescent entity; wherein the FRET hybridization probes hybridize adjacently to each other on the target nucleic acid; and wherein the spacer entities of the FRET hybridization probes are capable of forming non covalent interactions with each other in the presence of nucleic acid amplification primers, a template dependent nucleic acid polymerase, and deoxynucleoside triphosphates, in a buffer solution suitable for use in a template dependent nucleic acid amplification reaction under conditions suitable for amplification and hybridization of said hybridization probes to said target nucleic acid sequence; performing a template dependent nucleic acid amplification reaction; repeating steps a-d a predetermined number of times; then illuminating the sample with light at a frequency suitable for exciting the donor FRET moiety; then detecting either an increase in emission of light from said acceptor FRET moiety or a decrease in emission of light from said donor FRET moiety.

In another aspect, there is provided a method for performing a real-time template dependent nucleic acid amplification reaction, comprising: contacting a sample containing a target nucleic acid sequence with a pair of hybridization probes capable of hybridizing to a target nucleic acid sequence, each of said pair of FRET hybridization probe comprising a nucleotide sequence entity, said nucleotide sequence entity being substantially complementary to a portion of the sequence of the target nucleic acid; a fluorescent entity, said fluorescent entity being either a FRET donor entity or a FRET acceptor entity; and a spacer entity, said spacer entity connecting the nucleotide sequence entity and the fluorescent entity; wherein the FRET hybridization probes hybridize adjacently to each other on the target nucleic acid; and wherein the spacer entities of the FRET hybridization probes are capable of forming non covalent interactions with each other in the presence of nucleic acid amplification primers, a template dependent nucleic acid polymerase, and deoxynucleoside triphosphates, in a buffer solution suitable for use in a template dependent nucleic acid amplification reaction under conditions suitable for amplification and hybridization of said hybridization probes to said target nucleic acid sequence; performing a template dependent nucleic acid amplification reaction; repeating the previous steps a predetermined number of times; then illuminating the sample with light at a frequency suitable for exciting the donor FRET moiety; then detecting either an increase in emission of light from said acceptor FRET moiety or a decrease in emission of light from said donor FRET moiety.

In another aspect, there is provided a method for detecting a target nucleic acid sequence in a sample, comprising providing a pair of hybridization probes capable of hybridizing to a target nucleic acid sequence, each of said pair of FRET hybridization probe comprising a nucleotide sequence entity, said nucleotide sequence entity being substantially complementary to a portion of the sequence of the target nucleic acid; a fluorescent entity, said fluorescent entity being either a FRET donor entity or a FRET acceptor entity; and a spacer entity, said spacer entity connecting the nucleotide sequence entity and the fluorescent entity; wherein the FRET hybridization probes hybridize adjacently to each other on the target nucleic acid; and wherein the spacer entities of the FRET hybridization probes are capable of forming non covalent interactions with each other; contacting the target nucleic acid with the pair of hybridization probes, under conditions suitable for hybridization of the hybridization probes to the target nucleic acid sequence; and detecting the hybridization of the hybridization probes to the target nucleic acid sequence.

In another aspect, there is provided a reaction mixture for use in a dependent nucleic acid amplification reaction, comprising, in a solution a pair of hybridization probes capable of hybridizing to a target nucleic acid sequence, each of said pair of FRET hybridization probe comprising a nucleotide sequence entity, said nucleotide sequence entity being substantially complementary to a portion of the sequence of the target nucleic acid; a fluorescent entity, said fluorescent entity being either a FRET donor entity or a FRET acceptor entity; and a spacer entity, said spacer entity connecting the nucleotide sequence entity and the fluorescent entity; wherein the FRET hybridization probes hybridize adjacently to each other on the target nucleic acid; and wherein the spacer entities of the FRET hybridization probes are capable of forming non covalent interactions with each other; and at least one other component selected from the group consisting of nucleic acid amplification primers, a template dependent nucleic acid polymerase, deoxynucleoside triphosphates and a buffer suitable for use in a template dependent nucleic acid amplification reaction.

In another aspect, said spacer entities are nucleotide residues characterized in that the additional residues of the first hybridization probe may form base pairing interactions with the nucleotide residues of the second hybridization probe thus forming a stem structure, when hybridized to the target nucleic acid.

In contrast to standard FRET hybridization probes known in the art which are characterized in that the fluorescent compounds are in close proximity to the nucleotides forming the probe/target hybridization complex, the design of FRET hybridization probes as described herein avoids any potential interference of the fluorescent compounds with e.g. G residues that are present within the probe/target hybridization complex.

As used herein the term “substantially complementary” means that the respective sequences specifically hybridize to each other under standard annealing conditions. The length of the nucleotide sequence entity which is substantially complementary to the sequence of the target nucleic acid may vary between 10 and 40 nucleotide residues. Preferably, but depending on the AT content of the target nucleic acid, the length is between 15 and 30 nucleotide residues. At least, a perfect Watson Crick base pairing between more than 85% of the residues constituting the hybrid is required. In addition, a perfect complementarity over a segment of 10 constitutive nucleotide residues is required.

As used herein, the term “hybridizing adjacently” means that in case the two hybridization probes are hybridized to the target nucleic acid, there exists either no or only a small gap ranging over 0-10 and preferably 1-2 complementary nucleotide residues between the two probes with respect to the target nucleic acid sequence

In another aspect, a pair of FRET hybridization probes hybridizing adjacently to a target nucleic acid sequence are provided, each hybridization probe comprising
a nucleotide sequence entity which is substantially complementary to the sequence of the target nucleic acid
a fluorescent entity, the entity being either the FRET donor entity or the FRET acceptor entity; and
a spacer entity connecting the nucleotide sequence entity and the fluorescent entity, wherein due to the presence of the spacer entities, the intensity of fluorescence emission from the FRET donor entity and the intensity of fluorescence emission from the FRET acceptor entity are not substantially affected by any quenching activity of nucleotide residues either present in the sequence of the target nucleic acid or present in said nucleotide sequence entities of the hybridization probes.

As used herein, the term “not substantially affected” means that it is not possible with any conventional direct or indirect method to detect a quenching effect of more than 20% which is due to any G residue present in the target or the probes with regard to fluorescence emission of the FRET acceptor entity when the hybridization complex has been formed.

In other words, the intensity of fluorescence emission from the FRET acceptor entity of a pair of FRET hybridization probes as described herein, when hybridized to its target sequence is detectably increased compared to the intensity of fluorescence emission of a FRET acceptor entity of a comparative pair of FRET hybridization probes hybridized to the same target DNA, if the comparative pair of FRET hybridization probes is identical to the pair of hybridization probes with the exception that the comparative pair of hybridization probes does not comprise a spacer entity connecting the nucleotide sequence entity and the fluorescent entity.

As used herein, the term “detectably increased” means that a difference between the two pairs of FRET hybridization probes as described herein can be monitored in a conventional real time PCR assay, for example a LightCycler instrument (Roche Applied Sciences). Preferably, however, the detectable difference is more than 20%.

The degree of fluorescence signal increase depends significantly on the target nucleic acid sequence and on the pair of FRET dyes used. In certain cases the fluorescence signal increase can exceed 100%. When using quenching-sensitive dyes, more than 5-fold signal increases can be observed when comparing a pair of FRET hybridization probes as described herein with a pair of conventional FRET hybridization probes. Fluorescence signal increase is not only observed in conventional hybridization assays. As will be shown in the Examples, it is also detectable in real time PCR quantification and in melting curve analysis.

In general, the nucleotide residues carrying the spacer linked to the fluorescent moiety may be either internal, 5′ terminal or 3′ terminal residues, as long as upon hybridization of the pair of oligonucleotides to the target nucleic acid, fluorescence resonance energy transfer can take place when both FRET entities are brought into spatial vicinity such that subsequent to excitation of the FRET donor, fluorescence emission from the FRET acceptor can be monitored.

Preferably, however, one oligonucleotide carrying the first spacer and the first FRET entity is labeled at its 3′ terminal residue and the second oligonucleotide carrying the second spacer and the second FRET entity is labeled at its 5′ terminal residue such that when both oligonucleotides are hybridized to the target nucleic acid, the fluorescent labels of both FRET entities are brought in close vicinity to each other due to the fact that said terminal residues are base pairing to adjacent residues in the target nucleic acid or at least to residues which are only separated by one, two or at maximum less then 10 further residues.

In this context, it may be chosen arbitrarily which oligonucleotide carries the FRET donor moiety and which oligonucleotide carries the FRET acceptor moiety, since these elements can be introduced on both, either the 5′ or the 3′ end of an oligonucleotide by methods known in the art.

Further disclosed herein is a pair of FRET hybridization probes hybridizing adjacently to a target nucleic acid sequence, each hybridization probe comprising
a nucleotide sequence entity which is substantially complementary to the sequence of the target nucleic acid;
a fluorescent entity, the entity being either the FRET donor entity or the FRET acceptor entity; and
a spacer entity connecting the nucleotide sequence entity and the fluorescent entity, with the provisions that
due to the presence of the spacer entities, the intensity of fluorescence emission from the FRET donor entity and the intensity of fluorescence emission from the FRET acceptor entity is not substantially affected by any quenching activity of nucleotide residues either present in the sequence of the target nucleic acid or present in the nucleotide sequence entities of the hybridization probes, and
the spacer entities of the two members of the pair of FRET hybridization probes are capable of forming non covalent interactions with each other.

In another aspect, the non covalent interactions between the two members of a pair of FRET hybridization probes as disclosed herein, are nucleotide base pairing interactions and preferably A/T base pairing interactions, forming a stem structure.

It has been proven to be advantageous if the stem structure consists of two complementary single strands with equal numbers of nucleotide residues. However, it is also possible that the numbers of nucleotide residues is unequal, as long as upon hybridization, the two fluorescent moieties are brought in close vicinity to each other. This is usually the case, if the numbers of nucleotide residues only differ by 1 or 2. In another embodiment, the stem structure formed by the nucleotides generating the base pairing interactions optionally comprises single mismatches and/or nucleotide analogues and a basic linkers.

In order to avoid any effect of quenching due to nucleotide residues like G residues, each stem should comprise at least 1-3 additional base pairs and preferably A/T base pairs. On the other hand, introduction of 10 or more additional nucleotide residues on each spacer entity does not result in any improved effect, but on the other hand may lead to an undesired complex formation between the two oligonucleotides without binding to the target nucleic acid itself.

The first oligonucleotide is designed in such a way that 3′ to the nucleotide sequence entity which is substantially complementary to the sequence of the target nucleic acid, there are 1-10, preferably 3-8, and most preferably 3-5 additional nucleotide residues which act as a spacer entity. The 3′ terminal residue of these additional residues is labeled with the first FRET entity according to standard protocols known in the art. In one embodiment, the additional residues are A or T residues. In another embodiment, more than 60% of the additional residues are A or T residues. In yet another embodiment, the two or the three 3′ terminal residues are A or T residues.

Correspondingly, the second oligonucleotide is designed in such a way that 5′ to the nucleotide sequence entity which is substantially complementary to the sequence of the target nucleic acid, there are 1-10, preferably 3-8, and most preferably 3-5 additional nucleotide residues which act as a spacer entity and at the same time are capable of hybridizing to the spacer entity of the first oligonucleotide. The 5′ terminal residue of these additional residues is labeled with the second FRET entity again according to standard protocols known in the art. In one embodiment, the additional residues are A or T residues. In another embodiment, more than 60% of said additional residues are A or T residues. In yet another embodiment, the two or the three 3′ terminal residues are A or T residues.

In another aspect of the invention, the stem generated by the base pairing interactions contains one, two, three or at least less than five additional G/C base pairs. Although in this case it may be possible that the respective G residues of the stem may result in a quenching effect, the degree of quenching on different FRET hybridization probes may be comparable. However, strong fluorescence signal intensities are in general obtained where at least the one, two or three terminal base pairs forming the stem are A/T base pairs.

When both oligonucleotide probes are hybridized to the target nucleic acid, the fluorescent labels of both FRET entities are brought into close vicinity to each other but on the other hand are still separated over a certain distance from any potential G residue within either the target nucleic acid or G residues within that part of the hybridization probes which is substantially complementary to the target nucleic acid sequence.

In general, the design of hybridization probes described herein is applicable to any combination of fluorescent compounds, between which fluorescent energy transfer may take place. Illustratory examples which are not at all limiting the invention are fluorescein/Cy5 (Amersham, Inc., Piscataway, N.J.), fluorescein/LC-Red-640 (Roche Applied Science, Mannheim, Germany), fluorescein/LC-Red-705 (Roche Applied Science), and fluorescein/JA286 (EP 747 447).

In another aspect, the FRET acceptor entity may be a quencher moiety different from a fluorescent compound and consequently, decrease in fluorescence from the FRET donor moiety may be monitored. Examples for quencher compounds which may be used in this regard are Dabcyl (Kreuzer, K. A., et al., Clin Chem 47 (2001) 486-90) or so called Black Hole Quenchers (WO 01/86001)

Usually, the FRET hybridization probes are typical single stranded DNA molecules. Nevertheless, any kind of modification is possible. For example, the single stranded DNA may contain non-natural bases such as 7-deaza-purine, diamino-purine or C-nucleotides. The single stranded DNA may also have a modified sugar-phosphate backbone such as 2-O-methyl, phosphothioate, or anything similar.

The oligonucleotides acting as FRET hybridization probes may be labeled with the required fluorescent entity at any position by methods known in the art. For example, the oligonucleotides may be labeled internally at the nucleoside base or the phosphate moiety.

In one aspect, one oligonucleotide is labeled at the 5′ end and the second oligonucleotide is labeled at the 3′ end. Which oligonucleotide carries the FRET donor moiety and which oligonucleotide carries the FRET acceptor moiety may be chosen arbitrarily in this regard. Usually, the 5′ label may be introduced at the end of the oligonucleotide synthesis using an appropriate phosphoramidate carrying a fluorescent compound. Alternatively, after oligonucleotide synthesis, an oligonucleotide carrying a reactive amino group may be labeled with a fluorescent compound activated as an NHS ester. For the 3′ labeling of oligonucleotides, commercially available controlled pore glass particles may be used as a solid support for the start of a chemical oligonucleotide synthesis, which comprise a tri-functional spacer entity with a fluorescent compound. Examples for other spacer entities besides nucleotide residues that interact via non covalent interactions include those that involve all kinds of hydrogen bonding, for example polypeptide interactions, and all kinds of hydrophobic interactions, for example those based on-CF₂ groups and ionic attractions. Thus, spacers with non covalent interactions involve all kinds of hydrogen bonding (like in peptides/oligonucleotides) and all kinds hydrophobic interactions (like aryl-aryl, alkyl-alkyl interaction or attraction between fluorinated hydrocarbons). Ionic interaction can be used if one of the spacer is negatively charged and the other spacer is positively charged. In addition, it is possible that the spacer moiety is branched instead of being linear.

In another aspect, there may be compositions comprising the FRET hybridization probes disclosed above. More precisely, such a composition comprises a nucleic sample and a pair of FRET hybridization probes, wherein the pair of FRET hybridization probes hybridize adjacently to a target nucleic acid sequence, and each hybridization probe comprises (i) a nucleotide sequence entity which is substantially complementary to the sequence of the target nucleic acid, (ii) a fluorescent entity, the entity being either the FRET donor entity or the FRET acceptor entity, and (iii) a spacer entity connecting the nucleotide sequence entity and the fluorescent entity, wherein due to the presence of the spacer entity, the intensity of fluorescence emission from the FRET donor entity and the intensity of fluorescence emission from the FRET acceptor entity are not substantially affected by any quenching activity of nucleotide residues either present in the sequence of the target nucleic acid or present in the nucleotide sequence entities of the hybridization probes.

In another aspect, various methods and applications of using the oligonucleotide pairs and compositions disclosed above are provided. More precisely, a method directed to qualitative or quantitative detection of a nucleic acid sequence in a nucleic acid sample in provided, wherein said nucleic acid sample is being hybridized with a pair of FRET hybridization probes wherein said pair of FRET hybridization probes hybridizes adjacently to a target nucleic acid sequence, and each hybridization probe comprises (i) a nucleotide sequence entity which is substantially complementary to the sequence of the target nucleic acid, (ii) a fluorescent entity, the entity being either the FRET donor entity or the FRET acceptor entity, and (iii) a spacer entity connecting the nucleotide sequence entity and the fluorescent entity, wherein due to the presence of the spacer entity, the intensity of fluorescence emission from the FRET donor entity and the intensity of fluorescence emission from the FRET acceptor entity are not substantially affected by any quenching activity of nucleotide residues either present in the sequence of the target nucleic acid or present in the nucleotide sequence entities of the hybridization probe.

In one embodiment, such a method may be a typical hybridization assay. The hybridization may take place either in solution, or alternatively, either the target nucleic acid or one member of the pair of FRET hybridization probes already by immobilized on a solid support. The solid support itself, for example can be a hybridization membrane, a magnetic glass bead, a micro-array for immobilizing nucleic acids or any other suitable material known in the art.

In another embodiment, a part of the nucleic acid present in the sample is subjected to a nucleic acid amplification reaction prior or during the hybridization procedure, for example, a polymerase chain reaction (PCR). As a prerequisite, the target nucleic acid comprises a sequence substantially complementary or homologous to the sequence of the used hybridization probes. In other words, the hybridization probes described herein need to hybridize specifically to the part of the target nucleic acid which is being amplified.

In another aspect, there is disclosed a method, wherein a pair of FRET hybridization probes as described herein is used for monitoring the amplification of a target nucleic in real time. As it is known in the art, real time monitoring allows the generation of kinetic data and facilitates quantitative analysis. Thus, the present invention is also directed to a method of monitoring the amplification of a target nucleic acid by means of monitoring either the increase in fluorescence emission of the FRET acceptor entity or monitoring the decrease in fluorescence emission of the FRET donor entity during the amplification reaction itself.

Also disclosed herein are methods directed to the usage of the FRET hybridization probes disclosed above for melting curve analysis, wherein monitoring of the dissociation of a complex between a target nucleic acid and a hybridization probe allows for the detection of small sequence variants such as single nucleotide polymorphisms. More precisely, provided herein is a method for the determination of the melting profile of a hybrid consisting of a target nucleic acid and a pair of FRET hybridization probes according to the invention, characterized in that first, a ternary hybrid complex between the target nucleic acid and the two hybridization probes is formed. Subsequently, the temperature is increased and the thermal dissociation of the ternary complex is determined by means of monitoring fluorescence in real time. In other words, the method provides for the determination of the melting profile of a hybrid consisting of a target nucleic acid and a pair of FRET hybridization probes as disclosed above, characterized in that the fluorescence emission is determined as a function of temperature.

The design of the non covalent interactions between the two FRET hybridization probes and especially the selection of the number of A/T base pairing interactions may be used in order to obtain a pair of FRET hybridization probes with a distinct melting temperature. This is highly advantageous for the development of a multiplex assay comprising multiple pairs of hybridization probes in order to generate melting peaks which can unambigously be discriminated from each other.

In a last aspect, there is provided a kit comprising a pair of hybridization probes as disclosed above. Such a kit may comprise a pair of FRET hybridization probes as disclosed above. In addition, it may also contain oligonucleotides capable of acting as a primer pair for a nucleic acid amplification reaction.

Precisely, the kit comprises a pair of FRET hybridization probes consisting of a first oligonucleotide carrying a FRET donor entity and a second oligonucleotide carrying a FRET acceptor entity, wherein said pair of FRET hybridization probes hybridizes adjacently to a target nucleic acid sequence, and each hybridization probe comprises (i) a nucleotide sequence entity which is substantially complementary to the sequence of the target nucleic acid, (ii) a fluorescent entity, said entity being either the FRET donor entity or the FRET acceptor entity, and (iii) a spacer entity connecting said nucleotide sequence entity and said fluorescent entity, wherein due to the presence of the spacer entity, the intensity of fluorescence emission from said FRET donor entity and the intensity of fluorescence emission from said FRET acceptor entity are not substantially affected by any quenching activity of nucleotide residues either present in the sequence of said target nucleic acid or present in said nucleotide sequence entities of said hybridization probes. In addition, the kit may contain at least one additional component such as a nucleic acid polymerase, deoxynucleoside triphosphates or respective analogues and an appropriate buffer which may be used for a template dependent nucleic acid amplification reaction such as PCR. Furthermore, the kit may also comprise software tools such as compact discs carrying computer programs for quantitative analysis of relative or absolute nucleic acid quantification experiments.

The following Examples, references, sequence listing and Figures are provided to aid the understanding of the present invention, the true scope of which is set forth in the appended claims. It is understood that modifications can be made in the procedures set forth without departing from the spirit of the invention.

### EXAMPLES

### Example 1

Preparation of PCR Primers and Probes

Primers were synthesized on a 1 μmol scale on ABI (Foster City, Calif.) Model 394 synthesizer using commercially available standard phosphoramidites (DMTr ibu G, DMTr bzA; DMTr bz C and DMTr T) and the corresponding CPG support. The chemicals for standard synthesis were obtained from GlenResearch (Sterling, Va.). Removal of the oligonucleotides from the solid support and deprotection was carried out with 33% NH₃ for 8 h at 55° C. Synthesis was performed in the trityl on modus. Purification was done on a RP 18 Oligo R3 4.6×50 mm column (Perseptive Biosystems, Framingham, Mass.). Buffer A: 0.1M triethylammonium acetate in water pH 7.0/MeCN 95:5. Buffer B: MeCN. Gradient: 3 min 20% B; 12 min 12-40% B; flow rate 1 ml/min; detection 260 nm. Subsequently, the concentrated oligonucleotide solution was treated for 5 min with 80% acetic acid at room temperature in order to remove the 5′ DMTr protecting group. Afterwards, oligonucleotides were desalted with a RP 18 column and lyophilized in a Speed Vac.

5′ labeled oligonucleotide (JA 286/LC Red 640) synthesis was performed in the 1 μmol range. Commercially available standard phosphoramidites (DMTr ibu G, DMTr bzA; DMTr bz C and DMTr T) and chemicals for standard synthesis were obtained from Glen Research. The 5′ amino group was introduced by using commercially available 5′ amino modifier (Glen Research (cat no. 10-1916-90)). As solid support 3′ phosphate CPG (GlenResearch 20-2900-01) was used. Removal of the oligonucleotides from the solid support and deprotection was carried out with 33% NH₃ for 8 h at 55° C. The solution was evaporated under vacuum. The remainder was dissolved in 600 μl double distilled water and transferred in a microcentrifuge tube. 60 μl of sodium acetate buffer (3M, pH 8.5) were added. Upon addition of 1.8 ml ice cold ethanol the mixture was stored at −15° C. for 3 h. The solution was centrifuged at 10000×g for 15 min. The supernatant was decanted. The pellet was washed with 200 μl ice cold ethanol. After centrifugation the supernatant was decanted. The pellet was dissolved in 400 μl sodium borate buffer (0.1M pH 8.5) and was labeled according standard procedures.

NHS-activated LC-Red-640 and JA286 were used. NHS-LC-Red-640 is obtainable from Roche Applied Sciences (Cat. No: 2 015 161). NHS activated JA286 was synthesized according to EP 0747 447, example 1.

A solution of 1 mg of the dye NHS ester in DMF was added and reacted for 15 h The labeled oligonucleotide was purified by reversed phase chromatography, using a Oligo R3 4.6×50 mm column. Chromatography: buffer A: 0. 1M triethylammonium acetate in water pH 7.0. buffer B: 0.1 M triethylammonium acetate in water/MeCN 1:1. Gradient: 2 min 0% B in 45 min to 100% B (the gradient was stopped when a product started to elute). At 20-25% B the nonlabeled oligonucleotide eluted; at 60-65% B the desired labeled oligonucleotide eluted; at 100% B the dye eluted; flow rate was 1 ml/min, detection at 260 nm. The fractions from the labeled oligonucleotide peaks were collected and the solvent was removed by using a vacuum centrifuge. The remainder was dissolved in double distilled water and then evaporated again with vacuum centrifugation. This procedure was repeated three times. The pellet was dissolved in water and lyophilized.

3′ fluorescein labeled oligonucleotides were synthesized and purified according to the manufacturer's instructions in the package insert of the commercially available LightCycler Fluorescein CPG (Roche Applied Science cat no. 3138178)

### Example 2

Quantitative Real time PCR of Factor V DNA using a pair of FRET Hybridization Probes Labeled with Fluorescein/JA286

For amplification of a Factor V DNA fragment, a 20 μl Real Time PCR reaction mixture was set up as follows:
10⁶ or 10⁴ copies of a plasmid containing the Factor V gene (Gene Bank Accession No: M_{—}014335)
13 mM MgCl₂
500 nM each primers according to SEQ ID NO: 1 and 2
200 nM each FRET hybridization probes according to SEQ ID NO: 3 and 4 or 5 and 6, respectively.
PCR components of LightCycler DNA Master Hyb Probes Kit (Roche Applied Science, Cat. No. 2158825)

Primers and probes were used as follows:

SEQ ID NO: 1
Forward primer:
5′ GAG AGA CAT CGC CTC TGG GCT A
SEQ ID NO: 2
Reverse Primer
5′ TGT TAT CAC ACT GGT GCT AA
SEQ ID NO: 3
FRET donor probe
5′ AAT ACC TGT ATT CCT CGC CTG TC-Fluorescein
SEQ ID NO: 4
FRET acceptor probe
5′ JA286-AGG GAT CTG CTC TTA CAG ATT AGA AGT AGT CCT
ATT
SEQ ID NO: 5
FRET donor probe
5′ AAT ACC TGT ATT CCT CGC CTG TCA AAA A-Fluorescein
SEQ ID NO: 6
FRET acceptor probe
5′ JA286-TTT TTA GGG ATC TGC TCT TAC AGA TTA GAA GTA
GTC CTA TT

The probes according to SEQ ID NO: 3 and 5 were 3′ terminally labeled with fluorescein according to Example 1. The probes according to SEQ ID NO: 4 and 6 were 5′ terminally labeled with JA286 as a FRET acceptor according to Example 1. Since SEQ ID No: 5 comprises a 3′ terminal oligo-A pentamer, and SEQ ID NO: 6 comprises a 5′ terminal oligo-T pentamer, a respective FRET hybridization probe pair constitutes a pair of hybridization probes according to the invention.

Amplification was performed in a LightCycler instrument (Roche Applied Science) according to the following thermocycling protocol:

TABLE 1

| | Temp | Time | Ramp-rate | | |
|---|---|---|---|---|---|
| | [° C.] | [sec] | [° C./sec] | Acquisition | Cycles |
| Denaturation | 95 | 30 | 20.0 | none | 1 |
| Amplification | 95 | 0 | 20.0 | none | |
| | 55 | 10 | 20.0 | single | 45 |
| | 72 | 10 | 20.0 | none | |

Real time monitoring was performed using the 2^{nd} derivative threshold method over 45 cycles by measuring the fluorescence signals in a detection channel specific for JA286 emission (at 710 nm) and using arithmetic background correction for normalization of initial fluorescence background intensities.

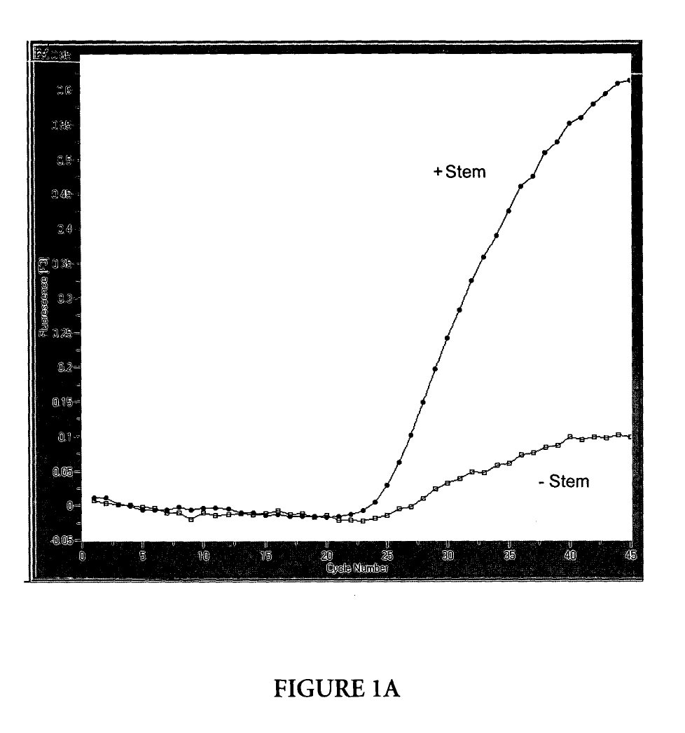

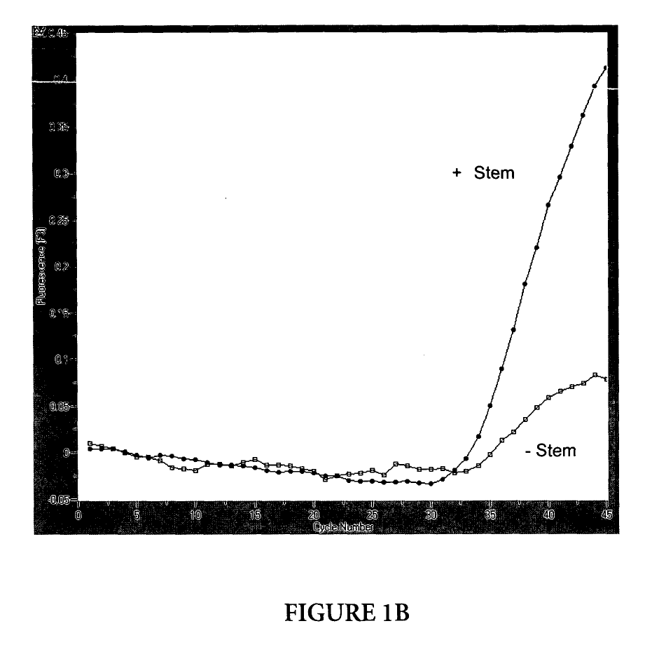

The result is shown in FIGS. 1A and 1B. As can be seen in the Figures, for both copy numbers of target DNA tested (FIG. 1A: 10⁶ copies, FIG. 1B: 10⁴ copies), usage of FRET hybridization probes comprising an AT stem resulted in significantly increased amplification signal over probes lacking an AT stem.

### Example 3

Melting Curve Analysis of Factor V DNA using a pair of FRET Hybridization Probes Labeled with Fluorescein/JA286

Subsequent to the reaction disclosed in Example 2, the samples were subjected to a melting curve analysis according to the instructions of the LightCycler manual (Roche Applied Sciences) using the following temperature transition protocol:

TABLE 2

| | Temp | Time | Ramp-rate | | |
|---|---|---|---|---|---|
| | [° C.] | [sec] | [° C./sec] | Acquisition | Cycles |
| Melting curve | 95 | 0 | 20.0 | none | |
| | 45 | 60 | 20.0 | continuous | 1 |
| | 75 | 10 | 0.1 | none | |
| Cooling | 40 | 30 | 20.0 | none | 1 |

Fluorescence monitoring was performed by measuring the absolute signal values obtained in the JA286 channel at 710 nm and subsequent calculation of the first derivative.

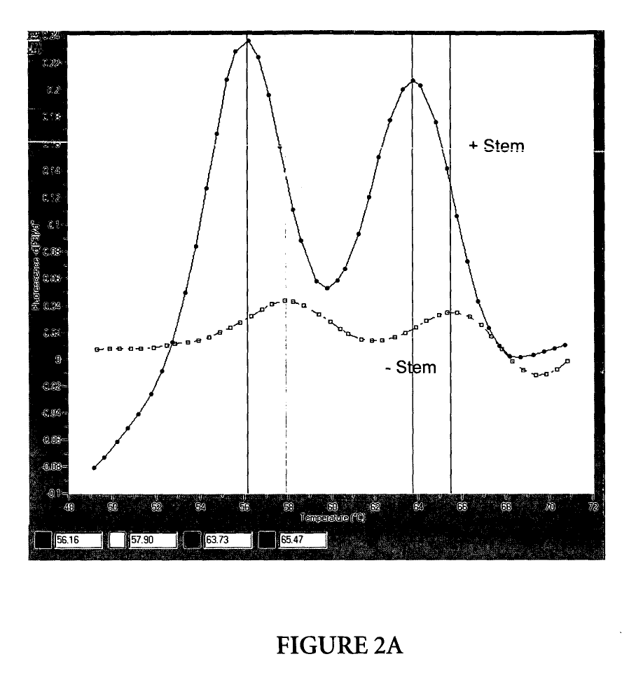

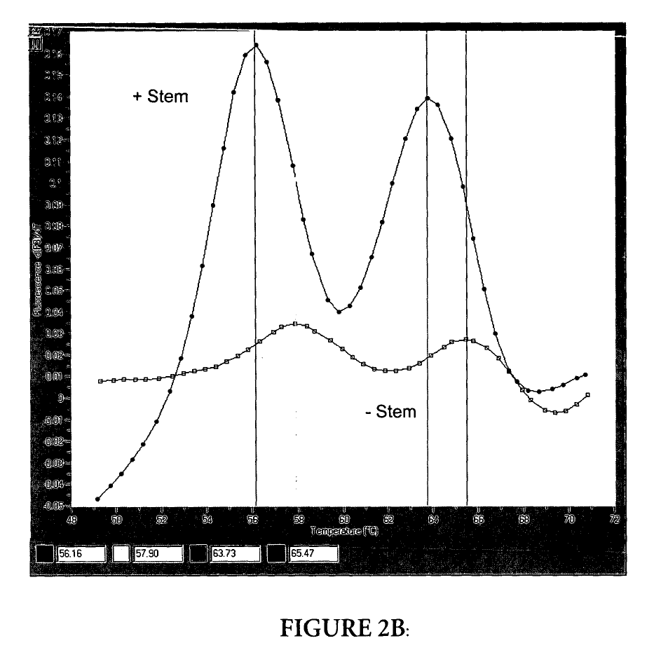

The result is shown in FIGS. 2A and 2B. As can be seen in the Figures, for both copy numbers of target DNA tested (FIG. 2A: 10⁶ copies, FIG. 2B: 10⁴ copies), usage of FRET hybridization probes comprising an AT stem resulted in significantly increased melting peaks over probes lacking an AT stem.

### Example 4

Quantitative Real time PCR of Factor V DNA using a pair of FRET Hybridization Probes Labeled with Fluorescein/LC-Red-640

Amplification of a Factor V DNA fragment was performed as in Example 2 with the exception that instead of JA286, LC-Red 640 was used as FRET acceptor moiety for 5′ terminal labeling of the hybridization probe according to SEQ ID NO: 4 and 6:

SEQ ID NO: 1
Forward primer:
5′ GAG AGA CAT CGC CTC TGG GCT A
SEQ ID NO: 2
Reverse Primer
5′ TGT TAT CAC ACT GGT GCT AA
SEQ ID NO: 3
FRET donor probe
5′ AAT ACC TGT ATT CCT CGC CTG TC-Fluorescein
SEQ ID NO: 4
FRET acceptor probe
5′ LCRED640-AGG GAT CTG CTC TTA CAG ATT AGA AGT AGT
CCT ATT
SEQ ID NO: 5
FRET donor probe
5′ AAT ACC TGT ATT CCT CGC CTG TCA AAA A-Fluorescein
SEQ ID NO: 6
FRET acceptor probe
5′ LCRED640-TTT TTA GGG ATC TGC TCT TAC AGA TTA GAA
GTA GTC CTA TT

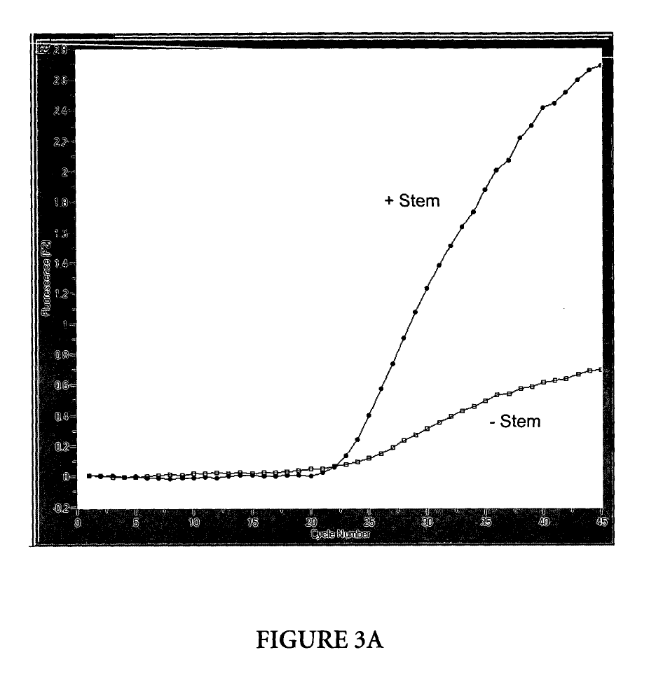

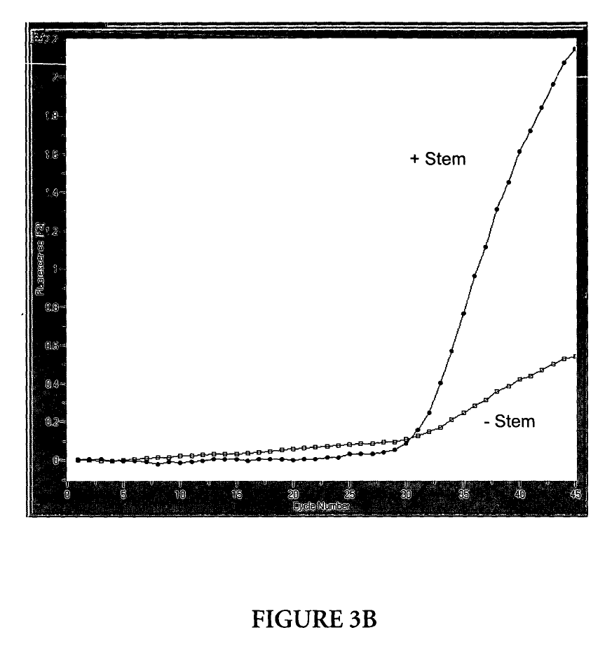

The result is shown in FIGS. 3A and 3B. As can be seen in the Figures, for both copy numbers of target DNA tested (FIG. 3A: 10⁶ copies, FIG. 3B: 10⁴ copies), the effect of an increased amplification signal using FRET hybridization probes comprising an AT stem (by using a FRET pair according to SEQ ID NOs: 5 and 6) could also be observed with a different FRET pair. It can be concluded that the positive effect of the FRET pair is independent from the type of dye pair which is actually used.

### Example 5

Melting Curve Analysis of Factor V DNA using a pair of FRET Hybridization Probes Labeled with Fluorescein/LC-Red-640

Subsequent to the reaction disclosed in Example 4, the samples were subjected to a melting curve analysis identical to the conditions disclosed in Example 3.

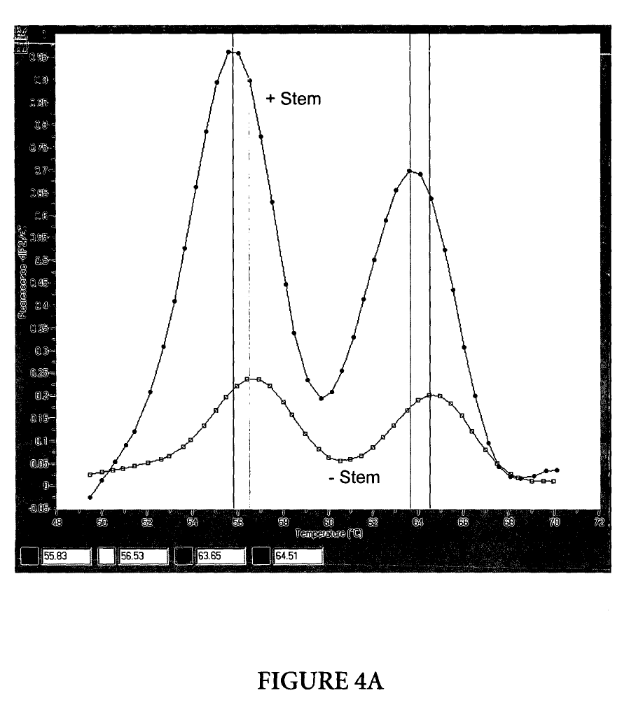

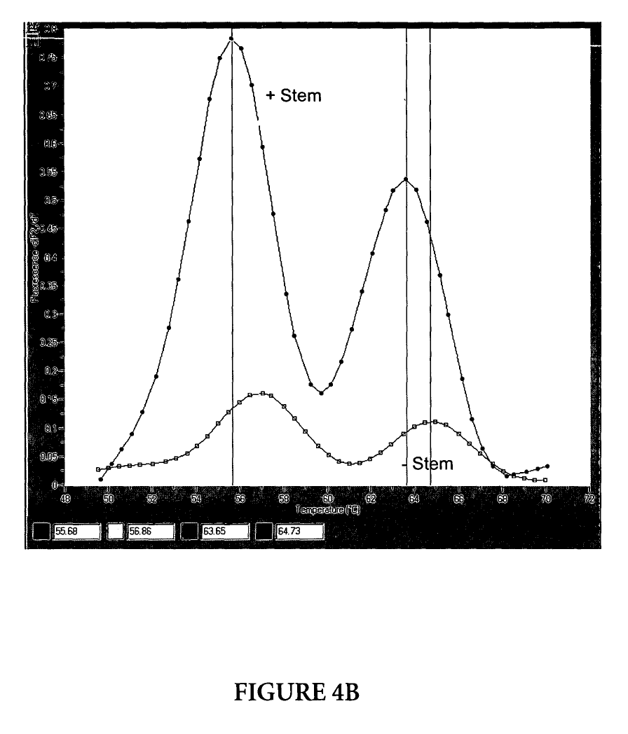

The result is shown in FIGS. 4A and 4B. As can be seen in the Figure, for both copy numbers of target DNA tested (FIG. 4A: 10⁶ copies, FIG. 4B: 10⁴ copies), usage of a FRET hybridization probes comprising an AT stem again resulted in a significant increase of melting peaks over FRET hybridization probes lacking an AT stem. Thus it can be concluded that improved melting curve analysis is independent from the type of dye pairs used.

### Example 6

Quantitative Real time PCR of G6PDH DNA using a pair of FRET Hybridization Probes Labeled with Fluorescein/JA286

For amplification of the G6PDH DNA (Gene Bank Acc. No: XM_{—}013149) fragment, conditions were identical as disclosed in Example 2, amplifying 10⁴ copies of target DNA with the following primers and probes:

SEQ ID NO: 7
Forward primer
GGG TGC ATC GGG TGA CCT G
SEQ ID NO: 8
Reverse Primer
5′ AGC CAC TGT GAG GCG GGA
SEQ ID NO: 9
FRET donor probe
5′ GGT GTT TTC GGG CAG AAG GCC ATC C-Fluorescein
SEQ ID NO: 10
FRET acceptor probe
5′ JA286-AAC AGC CAC CAG ATG GTG GGG TAG ATC TT
SEQ ID NO: 11
FRET donor probe
5′ GGT GTT TTC GGG CAG AAG GCC ATC CAA
AAA-Fluorescein
SEQ ID NO: 12
FRET acceptor probe
5′ JA286-TTT TTA ACA GCC ACC AGA TGG TGG GGT AGA TCT
T

In particular, 500 nM primers (each) according to SEQ ID NO: 7 and 8, and 200 nM FRET hybridization probes (each) according to SEQ ID NO: 9 and 11 or 10 and 12 respectively were used.

The probes according to SEQ ID NO: 9 and 11 were 3′ terminally labeled with fluorescein according to Example 1. The probes according to SEQ ID NO: 10 and 12 were 5′ terminally labeled with JA286. SEQ ID No: 11 comprises a 3′ terminal oligo-A pentamer, and SEQ ID NO: 12 comprises a 5′ terminal oligo-T pentamer, a respective FRET hybridization probe pair constitutes a pair of hybridization. Amplification was performed in a LightCycler instrument (Roche Applied Science) according to the following thermocycling protocol:

TABLE 3

| | Temp | Time | Ramp-rate | | |
|---|---|---|---|---|---|
| | [° C.] | [sec] | [° C./sec] | Acquisition | Cycles |
| Denaturation | 95 | 60 | 20.0 | none | 1 |
| Amplification | 95 | 0 | 20.0 | none | |
| | 55 | 15 | 20.0 | single | 45 |
| | 72 | 15 | 20.0 | none | |
| Cooling | 40 | 30 | 20.0 | none | 1 |

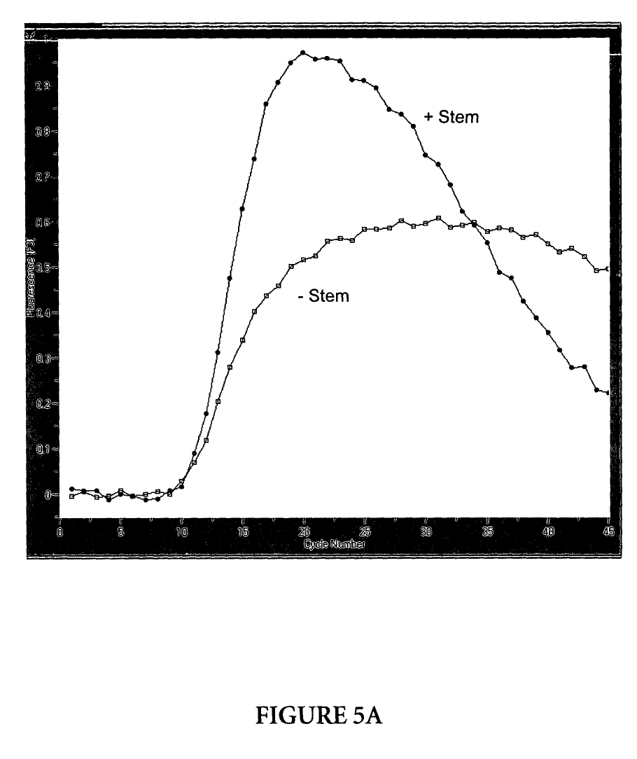

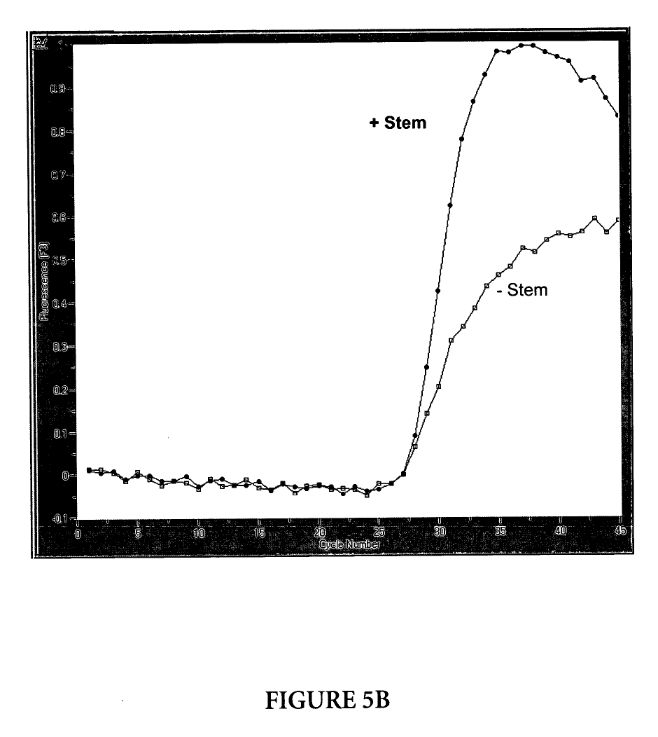

The result is shown in FIGS. 5A and 5B. As can be seen in the Figures, for both copy numbers of target DNA tested (FIG. 5A: 10⁸ copies, FIG. 5B: 10³ copies), usage of a FRET hybridization probe comprising an AT stem also resulted in a significantly increased amplification signal when another target DNA fragment was amplified. Thus it can be concluded that the FRET hybridization probes confer increased amplification signals independent from the type of target DNA to be amplified.

### Example 7

Quantitative real time PCR of Factor V DNA using FRET Hybridization Probes Labeled with Fluorescein/JA286 Comprising Different A/T Stems

The experiment was performed as disclosed in Example 2 with the modification that hybridization probes having no, one, three or five A/T base pair stems were tested. Thus, primers and probes were used as follows:

SEQ ID NO: 1
Forward primer:
5′ GAG AGA CAT CGC CTC TGG GCT A
SEQ ID NO: 2
Reverse Primer
5′ TGT TAT CAC ACT GGT GCT AA
SEQ ID NO: 3
FRET donor probe
5′ AAT ACC TGT ATT CCT CGC CTG TC-Fluorescein
SEQ ID NO: 4
FRET acceptor probe
5′ JA286-AGG GAT CTG CTC TTA CAG ATT AGA AGT AGT CCT
ATT
SEQ ID NO: 13
FRET donor probe 1A
5′ AAT ACC TGT ATT CCT CGC CTG TCA-Fluorescein
SEQ ID NO: 14
FRET acceptor probe 1T
5′ JA286-TAG GGA TCT GCT CTT ACA GAT TAG AAG TAG TCC
TAT T
SEQ ID NO: 15
FRET donor probe 3A
5′ AAT ACC TGT ATT CCT CGC CTG TCA AA-Fluorescein
SEQ ID NO: 16
FRET acceptor probe 3T
5′ JA286-TTT AGG GAT CTG CTC TTA CAG ATT AGA AGT AGT
CCT ATT
SEQ ID NO: 5
FRET donor probe 5A
5′ AAT ACC TGT ATT CCT CGC CTG TCA AAA A-Fluorescein
SEQ ID NO: 6
FRET acceptor probe 5T
5′ JA286-TTT TTA GGG ATC TGC TCT TAC AGA TTA GAA GTA
GTC CTA TT

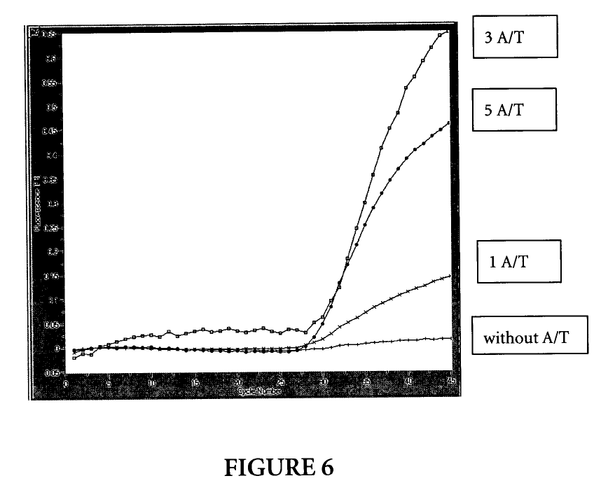

The result is shown in FIG. 6. As can be seen in the Figure, an improved amplification signal was already obtained with an A/T stem consisting of only one A/T base pair, the nucleotide residues of which do not hybridize to the target DNA. Moreover, the effect was significantly increased with A/T stems consisting of 3 or 5 A/T stems. Thus, it seems that the optimum length of an A/T stem according to the invention is between 3-5 base pairs.

### Example 8

Melting Curve Analysis of Factor V DNA using Hybridization Probes Labeled with Fluorescein/JA286 Comprising Different A/T Stems

Subsequent to the reaction disclosed in Example 7, the samples were subjected to a melting curve analysis as disclosed in Example 3.

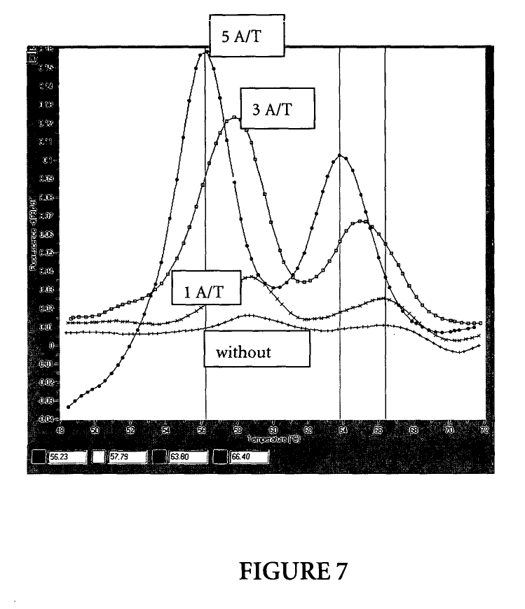

The result is shown in FIG. 7. As can be seen in the Figure, all FRET hybridization probes comprising an AT stem resulted in significantly increased melting peaks over FRET hybridization probes lacking an AT stem, even if the A/T stem was consisting of only one base pair.

List of References

Bernard, P. S., et al., Anal Biochem 255 (1998) 101-7.
Kreuzer, K. A., et al., Clin Chem 47 (2001) 486-90.
Seidel, C. A. M., et al., J Phys Chem 100 (1996) 5541-53
EP0747447
EP1046717
W00136668
W00173118
W00186001
W09746707
W09746712
W09746714

1. A composition comprising a pair of FRET hybridization probes hybridizing to a target nucleic acid sequence, each of said pair or FRET hybridization probe comprising
a nucleotide sequence entity, said nucleotide sequence entity being substantially complementary to a portion of the sequence of the target nucleic acid;
a fluorescent entity, said fluorescent entity being either a FRET donor entity or a FRET acceptor entity; and
a spacer entity, said spacer entity connecting the nucleotide sequence entity and the fluorescent entity;
wherein the FRET hybridization probes hybridize adjacently to each other on the target nucleic acid; and
wherein the spacer entities of the FRET hybridization probes are capable of forming non covalent interactions with each other.

2. A composition according to claim 1, wherein said non covalent interactions are nucleotide base pairing interactions.

3. A composition according to claim 2, wherein said nucleotide base pairing interactions are A/T base pairing interactions.

4. A composition according to claim 1, wherein said fluorescent entities on said pair of FRET hybridization probes are selected from the group consisting of fluorescein/Cy5, fluorescein/LC Red 640, fluorescein/LC Red 705, and fluorescein/JA286.

5. A composition according to claim 1, wherein said FRET acceptor entity is a Dabcyl or a Black Hole Quencher.

6. A composition according to claim 1, wherein at least one of the hybridization probes includes a nucleotide having a non-natural base.

7. A composition according to claim 6, wherein the non-natural base is selected from the group consisting of a 7-deazapurine, a diamino purine and a C-nucleotide.

8. A composition according to claim 1, wherein at least one of the hybridization probes includes a modified sugar-phosphate backbone.

9. A composition according to claim 8, wherein the modified sugar-phosphate backbone includes a 2-O methyl group or a phosphothioate.

10. A composition according to claim 1, wherein one of the hybridization probes is labeled at the 3′ terminal end and the other of the hybridization probes is labeled at the 5′ terminal end, such that upon hybridization of the probes to the target nucleic acid and excitation of the FRET donor entity, fluorescent resonance energy transfer to the FRET acceptor entity can occur.

11. A composition according to claim 1, wherein said non covalent interactions are hydrophobic interactions.

12. A composition according to claim 11, wherein said hydrophobic interactions are aryl/aryl, alkyl/alkyl or fluorinated hydrocarbons.

13. A composition according to claim 1, wherein said non covalent interactions are ionic interactions between a positively-charged group and a negatively-charged group.

14. A composition according to claim 1, wherein said spacer entity is branched.

15. A kit for use in performing a template dependent nucleic acid amplification reaction, comprising:
a pair of hybridization probes according to claim 1;
at least one other component selected from the group consisting of nucleic acid amplification primers, a template dependent nucleic acid polymerase, deoxynucleoside triphosphates and a buffer suitable for use in a template dependent nucleic acid amplification reaction; and
a container.

16. A method for performing a template dependent nucleic acid amplification reaction, comprising:
a) contacting a sample containing a target nucleic acid sequence with a pair of hybridization probes according to claim 1 in the presence of nucleic acid amplification primers, a template dependent nucleic acid polymerase, and deoxynucleoside triphosphates, in a buffer solution suitable for use in a template dependent nucleic acid amplification reaction under conditions suitable for amplification and hybridization of said hybridization probes to said target nucleic acid sequence;
b) performing a template dependent nucleic acid amplification reaction;
c) illuminating said sample with light at a frequency suitable for exciting the donor FRET moiety;
d) detecting either an increase in emission of light from said acceptor FRET moiety or a decrease in emission of light from said donor FRET moiety; and
e) repeating steps a-d a predetermined number of times.

17. The method of claim 16, wherein the template dependent nucleic acid amplification reaction is a polymerase chain reaction.

18. The method of claim 16, further comprising, after step e), changing the temperature of the sample and determining either an increase in emission of light from said acceptor FRET moiety or a decrease in emission of light from said donor FRET moiety as a function of temperature.

19. A method for performing a real-time template dependent nucleic acid amplification reaction, comprising:
a) contacting a sample containing a target nucleic acid sequence with a pair of hybridization probes according to claim 1 in the presence of nucleic acid amplification primers, a template dependent nucleic acid polymerase, and deoxynucleoside triphosphates, in a buffer solution suitable for use in a template dependent nucleic acid amplification reaction under conditions suitable for amplification and hybridization of said hybridization probes to said target nucleic acid sequence;
b) performing a template dependent nucleic acid amplification reaction;
c) repeating steps a-d a predetermined number of times; then
d) illuminating the sample with light at a frequency suitable for exciting the donor FRET moiety; then
e) detecting either an increase in emission of light from said acceptor FRET moiety or a decrease in emission of light from said donor FRET moiety.

20. The method of claim 19, wherein the template dependent nucleic acid amplification reaction is a polymerase chain reaction.

21. The method of claim 19, further comprising, after step e), changing the temperature of the sample and determining either an increase in emission of light from said acceptor FRET moiety or a decrease in emission of light from said donor FRET moiety as a function of temperature.

22. A method for detecting a target nucleic acid sequence in a sample, comprising:
a) providing a pair of hybridization probes according to claim 1;
b) contacting the target nucleic acid with the pair of hybridization probes, under conditions suitable for hybridization of the hybridization probes to the target nucleic acid sequence; and
c) detecting the hybridization of the hybridization probes to the target nucleic acid sequence.

23. The method of claim 22, wherein the hybridization probes and the target nucleic acid are in solution.

24. The method of claim 22, wherein the target nucleic acid sequence is bound to a solid support.

25. The method of claim 22, wherein one of said hybridization probes is bound to a solid support.

26. The method of claim 22, wherein said step of detecting is performed by illuminating the sample with light at a frequency suitable for exciting the donor FRET moiety; then detecting either an increase in emission of light from said acceptor FRET moiety or a decrease in emission of light from said donor FRET moiety.

27. A reaction mixture for use in a dependent nucleic acid amplification reaction, comprising, in a solution:
a pair of hybridization probes according to claim 1; and
at least one other component selected from the group consisting of nucleic acid amplification primers, a template dependent nucleic acid polymerase, deoxynucleoside triphosphates and a buffer suitable for use in a template dependent nucleic acid amplification reaction.


SEQUENCE LISTING (16 sequences)

SEQ ID NO: 1 - DNA, 22 bp, Artificial Sequence
  Description of Artificial Sequenceprimer
gagagacatcgcctctgggcta

SEQ ID NO: 2 - DNA, 20 bp, Artificial Sequence
  Description of Artificial Sequenceprimer
tgttatcacactggtgctaa

SEQ ID NO: 3 - DNA, 23 bp, Artificial Sequence
  Description of Artificial Sequenceprimer
aatacctgtattcctcgcctgtc

SEQ ID NO: 4 - DNA, 36 bp, Artificial Sequence
  Description of Artificial Sequenceprimer
agggatctgctcttacagattagaagtagtcctatt

SEQ ID NO: 5 - DNA, 28 bp, Artificial Sequence
  Description of Artificial Sequenceprimer
aatacctgtattcctcgcctgtcaaaaa

SEQ ID NO: 6 - DNA, 41 bp, Artificial Sequence
  Description of Artificial Sequenceprimer
tttttagggatctgctcttacagattagaagtagtcctatt

SEQ ID NO: 7 - DNA, 19 bp, Artificial Sequence
  Description of Artificial Sequenceprimer
gggtgcatcgggtgacctg

SEQ ID NO: 8 - DNA, 18 bp, Artificial Sequence
  Description of Artificial Sequenceprimer
agccactgtgaggcggga

SEQ ID NO: 9 - DNA, 25 bp, Artificial Sequence
  Description of Artificial Sequenceprimer
ggtgttttcgggcagaaggccatcc

SEQ ID NO: 10 - DNA, 29 bp, Artificial Sequence
  Description of Artificial Sequenceprimer
aacagccaccagatggtggggtagatctt

SEQ ID NO: 11 - DNA, 30 bp, Artificial Sequence
  Description of Artificial Sequenceprimer
ggtgttttcgggcagaaggccatccaaaaa

SEQ ID NO: 12 - DNA, 34 bp, Artificial Sequence
  Description of Artificial Sequenceprimer
tttttaacagccaccagatggtggggtagatctt

SEQ ID NO: 13 - DNA, 24 bp, Artificial Sequence
  Description of Artificial Sequenceprimer
aatacctgtattcctcgcctgtca

SEQ ID NO: 14 - DNA, 37 bp, Artificial Sequence
  Description of Artificial Sequenceprimer
tagggatctgctcttacagattagaagtagtcctatt

SEQ ID NO: 15 - DNA, 26 bp, Artificial Sequence
  Description of Artificial Sequenceprimer
aatacctgtattcctcgcctgtcaaa

SEQ ID NO: 16 - DNA, 39 bp, Artificial Sequence
  Description of Artificial Sequenceprimer
tttagggatctgctcttacagattagaagtagtcctatt